(19) United States
(12) Patent Application Publication
(10) Pub. No.: US 2026/0325006 A1
(43) Pub. Date: Oct. 22, 2026

(54) WEARABLE GAIT DISTURBANCE ANALYSIS APPARATUS

(71) Applicant: KOREA INSTITUTE OF SCIENCE AND TECHNOLOGY, Seoul (KR)

(72) Inventors: Choong Hyun KIM, Seoul (KR); Ji Su PARK, Seoul (KR)

(73) Assignee: KOREA INSTITUTE OF SCIENCE AND TECHNOLOGY, Seoul (KR)

(21) Appl. No.: 19/114,142

(22) PCT Filed: Feb. 24, 2023
(86) PCT No.: PCT/KR2023/002654

§ 371 Date: Mar. 21, 2025

(30) Foreign Application Priority Data
Sep. 22, 2022 (KR) 10-2022-0119888

(51) Int. Cl. A61B 5/103; A61B 5/00

(52) U.S. Cl. CPC A61B 5/1038; A61B 5/6807; A61B 5/7225; A61B 5/7275; A61B 5/742

(57) ABSTRACT

The present application relates to a wearable gait disturbance analysis apparatus that measures ground reaction forces, calculates the center of pressure (COPx), estimates the gait cycle, and determines gait disturbances using a polar coordinate system for intuitive assessment.

### TECHNICAL FIELD

The present application relates to a wearable gait disturbance analysis apparatus capable of evaluating whether a gait condition is good, i.e., gait soundness by estimating a pedestrian's gait cycle and analyzing a weight shift situation according to the estimated gait cycle.

### CROSS-REFERENCE TO RELATED APPLICATION

The present application claims priority to Korean Patent Application No. 10-2022-0119888, filed Sep. 22, 2022, the entire contents of which are hereby incorporated by this reference.

### EXPLANATION ON STATE-SUPPORTED RESEARCH AND DEVELOPMENT

This study was conducted under the support of the ‘Super-aging Response Disability Overcoming Research Project (Project Identification Number: 1711173293)’ of the Ministry of Science and ICT under the supervision of the Korea Institute of Science and Technology.

### BACKGROUND ART

Bipedal upright gait is the most fundamental activity of daily living unique to humans, and by examining a gait condition, it is possible to assess a human health status.

Gait is locomotion through space by patterned limb movements, and maintaining balance is essential for normal gait. However, since this balance becomes difficult to maintain, a state in which the normal gait is impossible is called gait disturbance.

Recently, as interest in personal health management has increased, many studies have been conducted on the effects of these gait characteristics on health or on gait characteristics that change according to health status. In other words, according to the existing studies, efforts are being made to determine the health status or whether there is disease through analysis of gait patterns and evaluation of gait soundness.

Since people have different physical characteristics, the gait patterns also appear in various ways. The existing inventions have evaluated gait balance by simply comparing bio-signals acquired according to the gait using a pressure sensor or an acceleration sensor, but had difficulty distinguishing between cases where patient's gait disturbance is mild or when healthy adults exhibited slight abnormality in gait. In addition, when diagnosing gait disturbance of patients with gait disturbance using a center of pressure (COP) in a direction of gait progression and a direction perpendicular thereto, that is, both COPy and COPx, the lack of consistency in changes in COPy leads to a significantly higher rate of determination errors.

### RELATED ART DOCUMENT

### Patent Document

Korean Patent Publication No. 10-1648270

### DISCLOSURE

### Technical Problem

Accordingly, the present application was conceived to solve the above-described problem, and detects the phenomenon in which a body weight is excessively shifted to one of left and right feet during walking using a gait disturbance analysis technology using an insole-based wearable device.

In the technology of this application, a gait cycle is accurately analyzed using only a medial-lateral component (COPx) of a human body which is relatively less affected by a degree of gait disturbance among positions of a center of pressure (COP) in the human body.

The gait cycle and the change in a position of COPx according to the gait cycle are displayed in polar coordinates, and a weight support ratio of left and right feet stances during walking is compared through a drawing to intuitively detect whether there is gait disturbance and present a quantified standard necessary for objective gait disturbance determination.

### Technical Solution

To achieve the above-described technical problems, the present application provides a wearable gait disturbance analysis apparatus, including: a plurality of left sole sensor units that measure a left ground reaction force (GRF) signal generated from a left sole during walking; a plurality of right sole sensor units that measure a right ground reaction force signal generated from a right sole during walking; and a calculation unit that receives and calculates the left ground reaction force signal and the right ground reaction force signal, in which the calculation unit may integrate the left ground reaction force signal and the right ground reaction force signal to calculate a center of pressure x-coordinate (COPx) in a human body in a medial-lateral direction of the human body, and use the calculated COPx to estimate a gait cycle.

In an embodiment, the calculation unit may calculate a change in the COPx according to the estimated gait cycle, and the wearable gait disturbance analysis apparatus may further include a gait disturbance diagnosis unit that diagnoses gait disturbance based on the change in the COPx.

In an embodiment, a display unit may display the change in the COPx according to the gait cycle in polar coordinates.

In an embodiment, the calculation unit may compare areas within two closed curves on upper and lower sides, respectively, displayed on the display unit and calculate a slope of a tangent line at a contact point between the two closed curves.

In an embodiment, the polar coordinate may be represented by the closed curve on the upper side where the COPx is a positive value and the closed curve on the lower side where the COPx is a negative value, and each of the closed curve on the upper side and the closed curve on the lower side may correspond to one of a right foot stance and a left foot stance.

In an embodiment, the calculation unit may calculate an area of the closed curve on the upper side and an area of the closed curve on the lower side, the gait disturbance diagnosis unit may diagnose the gait disturbance when a difference between the area of the closed curve on the upper side and the area of the closed curve on the lower side is greater than or equal to a preset standard, and when the gait disturbance diagnosis unit diagnoses the gait disturbance, it may be determined that a foot corresponding to the larger area of the two closed curves touches the ground longer than the other foot during walking and supports a body weight for a longer time.

In an embodiment, the calculation unit may calculate a slope of a tangent line at a contact point between the closed curve on the upper side and the closed curve on the lower side, and the gait disturbance diagnosis unit may diagnose the gait disturbance when the slope of the tangent line is greater than or equal to a preset standard.

In an embodiment, the calculation unit may calculate an area ratio index (ARI), which is a value representing a difference in time during which each foot supports the body weight as a ratio, using the following [Equation 1], [Equation 2], [Equation 3], and [Equation 4], and the gait disturbance diagnosis unit may diagnose whether there is the gait disturbance using the ARI as an index.

$${Area}_{Closed} = \int \frac{1}{2} {r}^{2} θ d θ \qquad [ Equation 1 ]$$

$${Area}_{ratio . R} = ( {Area}_{Closed . R} / ( {Area}_{Closed . R} + {Area}_{Closed . L} ) ) / 100 \qquad [ Equation 2 ]$$

$${Area}_{ratio . L} = ( {Area}_{Closed . L} / ( {Area}_{Closed . R} + {Area}_{Closed . L} ) ) / 100 \qquad [ Equation 3 ]$$

$$A R I = \vert {Area}_{ratio . Ratio . L} - {Area}_{ratio . L} \vert \qquad [ Equation 4 ]$$

The Area_{Closed} may be an area within a closed curve, the Area_{Closed.R} and Area_{Closed.L} may be an area of a closed curve corresponding to the right foot stance and an area of a closed curve corresponding to the left foot stance, respectively, the Area_{ratio.R} may be a ratio of the area of the closed curve corresponding to the right foot stance to a total area of the two closed curves, and Area_{ratio.L} may be a ratio of the area of the closed curve corresponding to the left foot stance to the total area of the two closed curves.

In an embodiment, the gait disturbance diagnosis unit may determine that there is the gait disturbance when the ARI value is greater than or equal to a preset standard.

In an embodiment, the COPx in the human body may be viewed in real time through a smart device.

In an embodiment, the wearable gait disturbance analysis apparatus may further include an amplifier that amplifies the left ground reaction force signal and the right ground reaction force signal.

In an embodiment, the wearable gait disturbance analysis apparatus may further include a timer that synchronizes the left ground reaction force signal and the right ground reaction force signal.

In an embodiment, the plurality of left sole sensor units and the plurality of right sole sensor units may be installed on a big toe, a 1st metatarsal bone, a 5th metatarsal bone, a cuboid bone, and a heel of the left and right feet, respectively.

In an embodiment, the ARI may be utilized as a biomarker for diagnosing a patient with the gait disturbance.

### Effect of the Invention

According to the present application, by acquiring and analyzing ground reaction force information generated during walking using a pressure sensor installed on a bottom of an insole, it is possible to understand a shift situation of a center of pressure in a human body in real time. In addition, the pressure sensor installed on the bottom of the insole is designed to be a low-power consumption type, so it can acquire bio-data for a long time, and can be applied all the time regardless of time and place.

The present application determines gait soundness of a wearer and directly provides analysis information to a PC or smart device in real time, so it can be utilized as a low-cost, high-efficiency health management system. The present application relates to a telemedicine device that measures patient's bio-data and remotely transmits the measured patient's bio-data so that medical staff can examine the patient. The acquired data can be built into big data and utilized for the advancement of gait disturbance analysis technology in the future.

The present application displays a gait cycle and a change in a position of COPx in the polar coordinates, compares a weight support ratio of left and right feet stances during walking through drawing to intuitively detect whether there is gait disturbance, and presents a quantified standard necessary for objective gait disturbance determination. Therefore, it is easy to compare or distinguish gait characteristics of healthy adults and patients with gait disturbance, and it is possible to distinguish between an affected foot and a healthy foot.

In addition, according to the present application, by developing and applying a personal wearable gait analysis apparatus, it is possible to provide a low-cost health management system that allows anyone to easily analyze their own gait patterns and use the analyzed gait patterns as health information.

### DESCRIPTION OF DRAWINGS

FIG. 1 is a schematic diagram illustrating a wearable gait disturbance analysis apparatus according to an embodiment of the present application.

FIG. 2 is a flowchart illustrating an operation process of the wearable gait disturbance analysis apparatus according to an embodiment of the present application.

FIG. 3 is a schematic diagram describing a configuration of the wearable gait disturbance analysis apparatus according to an embodiment of the present application.

FIG. 4 is a schematic diagram describing an attachment location of a sensor of the wearable gait disturbance analysis apparatus according to an embodiment of the present application.

FIG. 5 is a diagram illustrating a process of estimating a gait cycle using the wearable gait disturbance analysis apparatus and a frequency adaptive oscillator according to an embodiment of the present application.

FIG. 6 is a graph illustrating a gait cycle of a pedestrian acquired by wearing the wearable gait disturbance analysis apparatus according to an embodiment of the present application and a change in a center of pressure x-coordinate (COPx) in a medial-lateral direction of the human body according to the gait cycle in orthogonal coordinates.

FIG. 7 is a graph illustrating the gait cycle of the pedestrian acquired by wearing the wearable gait disturbance analysis apparatus according to an embodiment of the present application and the change in the center of pressure x-coordinate (COPx) in the medial-lateral direction of the human body according to the gait cycle in polar coordinates.

FIG. 8A is a graph illustrating a gait cycle of a healthy adult acquired by wearing the wearable gait disturbance analysis apparatus according to an embodiment of the present application and the change in the center of pressure x-coordinate (COPx) in the medial-lateral direction of the human body according to the gait cycle in the polar coordinate.

FIG. 8B is a graph illustrating a gait cycle of a patient with gait disturbance acquired by wearing the wearable gait disturbance analysis apparatus according to an embodiment of the present application and the change in the center of pressure x-coordinate (COPx) in the medial-lateral direction of the human body according to the gait cycle in the polar coordinates.

### MODE FOR DISCLOSURE

Hereinafter, a wearable gait disturbance analysis apparatus and a gait analysis method using the same according to an embodiment of the present application will be described with reference to the attached drawings, through a preferred embodiment of the present application.

Before proceeding with the description, when it is stated that a certain part “includes” a certain component, unless specifically stated otherwise, it means further including other components rather than excluding other components.

Furthermore, although the embodiments of the present application have been described with reference to the accompanying drawings, this is for illustrative purposes only, and does not limit the technical concept, structure, and application of the present application.

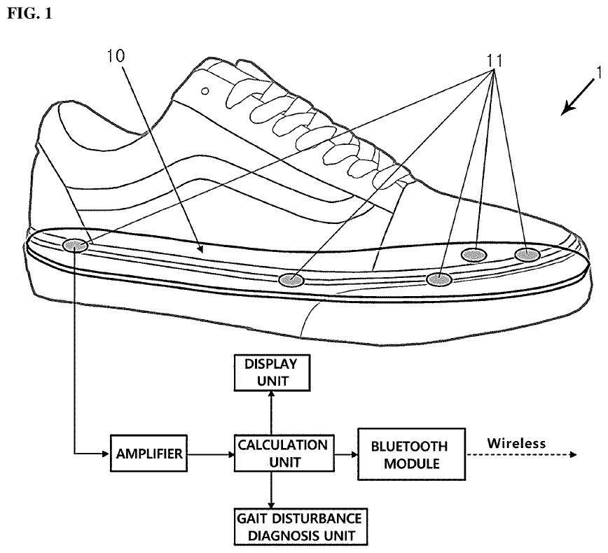

FIG. 1 is a schematic diagram illustrating a wearable gait disturbance analysis apparatus 1 according to an embodiment of the present application.

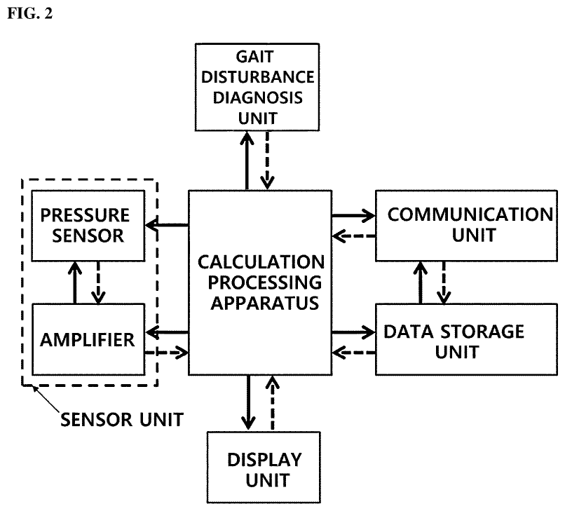

FIG. 2 is a flowchart illustrating an operation process of the wearable gait disturbance analysis apparatus 1 of FIG. 1 according to an embodiment of the present application.

In general, soles of both feet of a human body alternately and repeatedly touch and leave the ground during walking. Therefore, the wearable gait disturbance analysis apparatus 1 according to an embodiment of the present application may measure and compare changes in ground reaction force that appears on both soles for each gait step, thereby distinguishing each gait step and evaluating gait soundness.

Specifically, referring to FIG. 1 and FIG. 2 together, the wearable gait disturbance analysis apparatus 1 according to an embodiment of the present application includes a sensor 11 that measures ground reaction force that occurs on a sole of a foot of a human body during walking and an amplifier, and may include a calculation unit that performs calculation using ground reaction force data and synchronizes a measurement time of both feet data, a storage unit that stores the measured ground reaction force data, and a communication unit that transmits ground reaction force data of both feet that integrates left ground reaction force data and right ground reaction force data to a computer, a smart device, etc. Additionally, the wearable gait disturbance analysis apparatus 1 may further include a gait disturbance diagnosis unit. The calculation unit calculates a change in a center of pressure x-coordinate (COPx) in a human body in a medial-lateral direction of the human body, and the gait disturbance diagnosis unit diagnoses gait disturbance based on the change in the COPx. The wearable gait disturbance analysis apparatus 1 may further include a display unit that displays the change in the COPx according to the gait cycle in polar coordinates.

In addition, as illustrated in FIG. 1, the wearable gait disturbance analysis apparatus 1 according to an embodiment of the present application may include a plurality of sensors 11 attached to a bottom of an insole 10 inside a shoe, and in the present embodiment, five sensors may be used for each of both feet to measure the ground reaction force data of both feet.

In addition, the measured left ground reaction force signal and right reaction force signal may be amplified through the amplifier and then combined in the calculation unit.

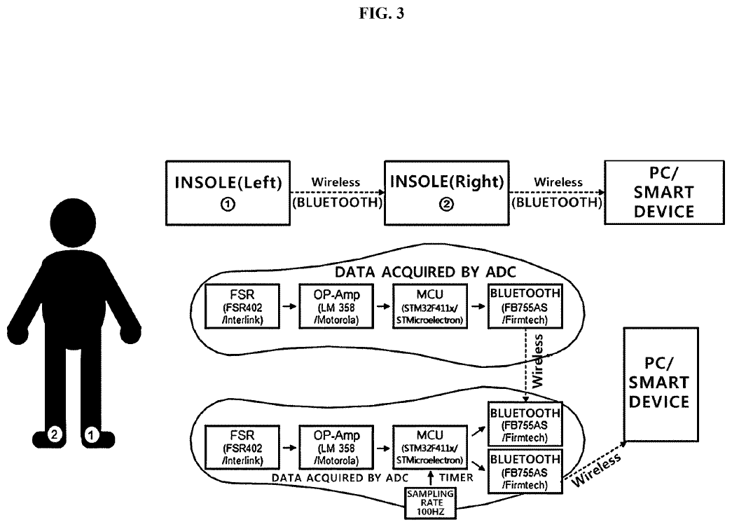

FIG. 3 is a schematic diagram describing a configuration of the wearable gait disturbance analysis apparatus 1 according to an embodiment of the present application.

As illustrated in FIG. 3, the calculation unit may integrate the left ground reaction force signal and the right ground reaction force signal to calculate the center of pressure x-coordinate (COPx) in the human body in the medial-lateral direction of the human body, and then use the calculated COPx to estimate a gait cycle.

In addition, the gait disturbance diagnosis unit may diagnose the gait disturbance based on the change in the COPx according to the estimated gait cycle.

These results may be transmitted to a computer or smart device for gait analysis via a Bluetooth device to view data in real time, and a separate timer may be set to synchronize the ground reaction force data of both feet using the time data.

A printed circuit board (PCB) of the bottom of the insole 10 inside the shoe of the wearable gait disturbance analysis apparatus 1 and a data collection method may be found in FIG. 3. The PCB may be manufactured small and light in order to minimize the effect on a user during walking. In an embodiment, a microcontroller unit (MCU) that performs data collection and communication may use the STM32F411x developed by STMicroelectronics. The ground reaction force may be measured using a force sensing resistor (FSR 402, Interlink Electronics, Inc. CA 93012, USA), and the ground reaction force data collected by a left foot MCU may be transmitted to the right foot MCU and then synchronized with the ground reaction force data of the right foot and combined, and then finally transmitted to and/or collected by a PC. In one example, a sampling rate of the ground reaction force may be 100 Hz.

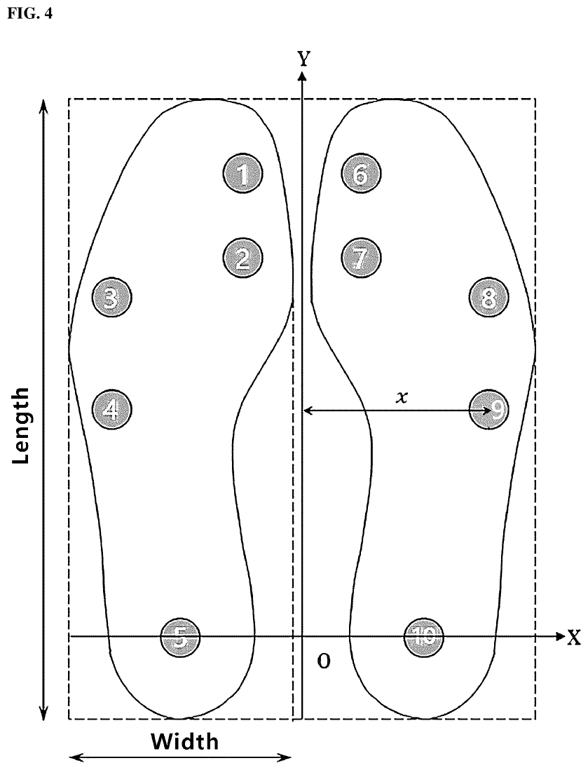

FIG. 4 is a schematic diagram describing an attachment location of the sensor 11 of the wearable gait disturbance analysis apparatus 1 according to an embodiment of the present application.

As illustrated in FIG. 4, in an embodiment of the present application, the attachment locations of the pressure sensor 11 are a total of five locations such as a big toe, a 1st metatarsal bone, a 5th metatarsal bone, a cuboid bone, and a heel, which sequentially touch the ground for each gait step.

As described in FIG. 4, a line segment connecting centers of heels of both feet is represented as an x-axis. In the existing invention, when using the COP for estimating the gait cycle, both COP values in a direction of gait progression and a direction perpendicular thereto, i.e., COPy and COPx, were used. However, in the case of patients with the gait disturbance, there was a problem that the lack of consistency in a change in COPy leads to a significantly higher rate of determination errors. Therefore, in the present application, COPx data, which is less affected by the gait disturbance is applied as input data to an adaptive frequency oscillator (AO) algorithm of the calculation unit to estimate the gait cycle, and then the change in COPx is examined in one gait cycle, thereby distinguishing between healthy adults and patients with gait disturbance.

Here, the COPx may be obtained using the following [Equation 1].

$$C O {P}_{x} = \frac{\sum ( {F}_{i} \times {x}_{i} )}{\sum {F}_{i}} \times \frac{100}{{x}_{5 th - meta}} \qquad [ Equation 1 ]$$

xᵢ is a position of an i-th FSR and is defined based on a height of a wearer. Fᵢ is a magnitude of the ground reaction force signal measured at an i-th FSR. In addition, in order to evaluate the COPx as a ratio to the outermost sensor position of the foot, the COPx value may be obtained by being divided by the position of the FSR positioned at the 5th metatarsal bone of the right foot.

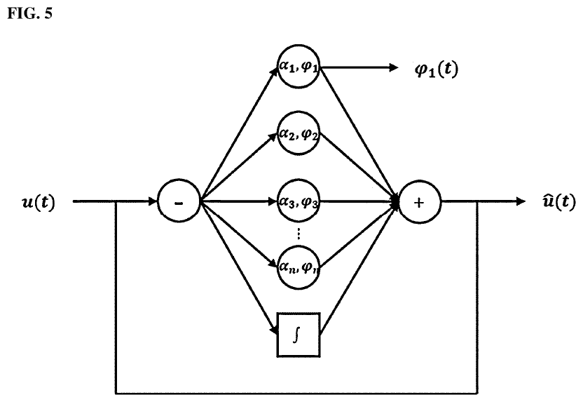

FIG. 5 is a diagram illustrating a process of estimating the gait cycle using the wearable gait disturbance analysis apparatus and the frequency adaptive oscillator according to an embodiment of the present application.

Referring to FIG. 5, AO is a method of obtaining an estimated signal û (t) based on the assumption that an input signal u (t) is composed of a linear combination of signals composed of a certain frequency and multiples of the frequency. The [Equation 2] used is as follows.

\dot{φ}ᵢ(t)=iω(t)+k_{φ}e(t)cos(φᵢ(t))

\dot{ω}(t)=k_{ω}e(t)cos(φ₁(t))

\dot{α}ᵢ(t)=k_{α}e(t)sin(φᵢ(t))

α₀k₀e(t)

û(t)=α₀(t)+Σαᵢ(t)sin(φᵢ(t))

e(t)=u(t)−û(t) [Equation 2]

Looking at the variables defined in the above [Equation 2], φᵢ and aᵢ are the phase and amplitude of an i-th oscillator, respectively, a₀ is the magnitude of the basic offset of the signal, and ω is the frequency of the signal. AO calculates a trained signal û using these four variables. The AO algorithm of the calculation unit may be designed as a 5th-order equation, a sixth-order equation, etc. (i=5, 6). Here, k_{φ}, k_{ω}, k_{α}, and k₀ are parameters that determine the speed at which the phase, amplitude, and frequency adapt to the input signal. The calculation unit may obtain the estimated signal φᵢ as the final gait cycle.

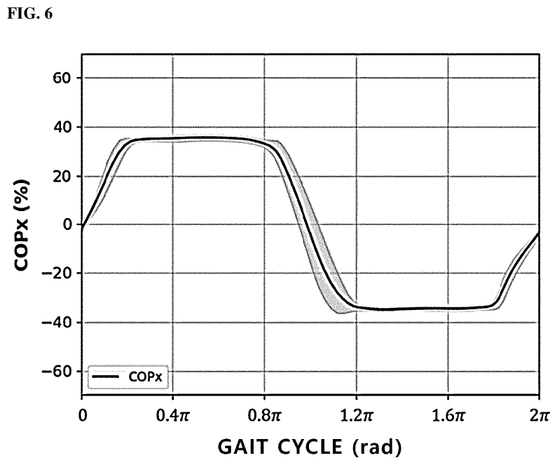

FIG. 6 is a graph illustrating the gait cycle of the pedestrian acquired by wearing the wearable gait disturbance analysis apparatus according to an embodiment of the present application and a trajectory (i.e., COPx, φᵢ) of the change in the center of pressure x-coordinate (COPx) in the medial-lateral direction of the human body according to the gait cycle in orthogonal coordinates.

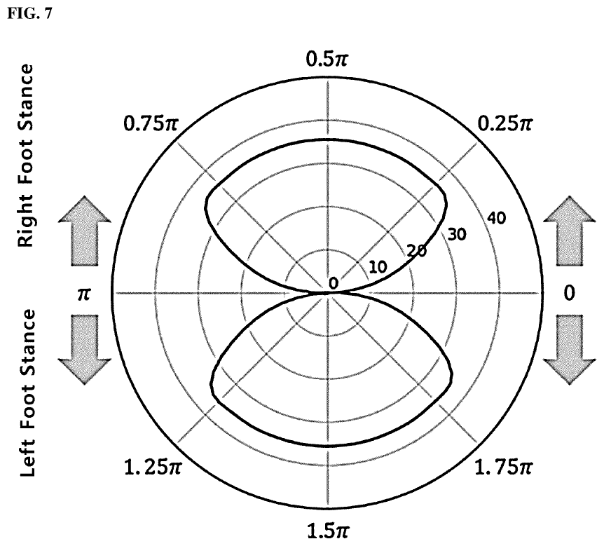

FIG. 7 is a graph illustrating the gait cycle of the pedestrian acquired by wearing the wearable gait disturbance analysis apparatus according to an embodiment of the present application and the trajectory (i.e., COPx, φᵢ) of the change in the center of pressure x-coordinate (COPx) in the medial-lateral direction of the human body according to the gait cycle in the polar coordinate.

Referring to FIG. 7, the orthogonal coordinate graph of FIG. 6 may be converted into the polar coordinate using the following [Formula 3].

r=|COPx|

θ=φ₁ [Equation 3]

The closed curves on the upper and lower sides of FIG. 7 correspond to the right foot stance and the left foot stance, where the COPx is a positive value and a negative value, respectively.

The calculation unit may compare areas within two closed curves on upper and lower sides, respectively, displayed on the display unit and calculate a slope of a tangent line at a contact point.

The gait disturbance diagnosis unit may diagnose the gait disturbance when the difference between the area of the closed curve on the upper side and the area of the closed curve on the lower side is greater than or equal to a preset standard. When the gait speed is constant, in an ideal gait condition, the sum of the areas enclosed by the two closed curves in each gait cycle should always be constant, and the areas of the two closed curves are the same, so the time for both feet to touch the ground is the same. However, in the actual gait, the areas of the two closed curves are different from each other. When the area of the closed curve on the upper side is larger than that of the closed curve on the lower side, it means that the subject touches his/her right foot on the ground longer than his/her left foot during walking, which may mean that the right foot supports the body weight for a longer time.

The calculation unit calculates an area ratio index (ARI), which is a value that represents the difference in the time each foot supports the body weight as a ratio, using the following [Equation 4], and the gait disturbance diagnosis unit may diagnose whether there is the gait disturbance using the ARI as an index.

$${Area}_{Closed} = \int \frac{1}{2} {r}^{2} θ d θ \qquad [ Equation 4 ]$$

$${Area}_{ratio . R} = ( {Area}_{Closed . R} / ( {Area}_{Closed . R} + {Area}_{Closed . L} ) ) / 100$$

$${Area}_{ratio . L} = ( {Area}_{Closed . L} / ( {Area}_{Closed . R} + {Area}_{Closed . L} ) ) / 100$$

$$A R I = \vert {Area}_{ra tio . R} - {Area}_{ratio . L} \vert$$

The Area_{Closed} may be an area within a closed curve, the Area_{Closed.R} and Area_{Closed.L} may be an area of a closed curve corresponding to the right foot stance and an area of a closed curve corresponding to the left foot stance, respectively, the Area_{ratio.R} may be a ratio of the area of the closed curve corresponding to the right foot stance to a total area of the two closed curves, and Area_{ratio.L} may be a ratio of the area of the closed curve corresponding to the left foot stance to the total area of the two closed curves. The gait disturbance diagnosis unit may determine that there is the gait disturbance when the ARI value is greater than or equal to the preset standard. Patients with gait disturbance include stroke patients as well, and the gait disturbance in the stroke patient, which is one of the major complications that restricts activities of daily living, requires long-term rehabilitation, and the stroke patients suffer from hemiplegic gait with gait asymmetry after rehabilitation. In an embodiment, the ARI may be utilized as a biomarker for diagnosing patients with gait disturbance such as the stroke patient.

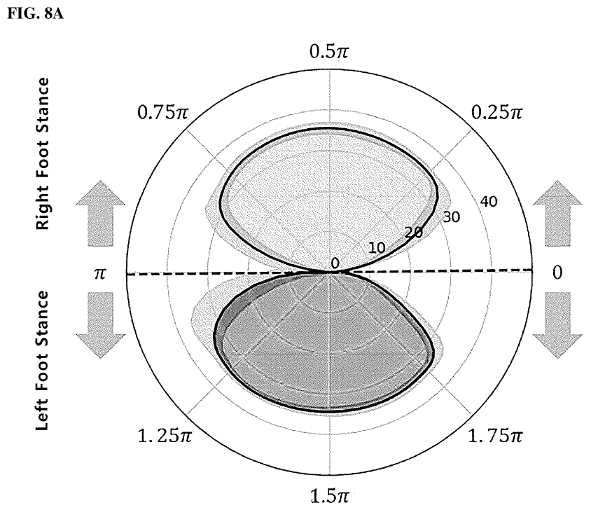

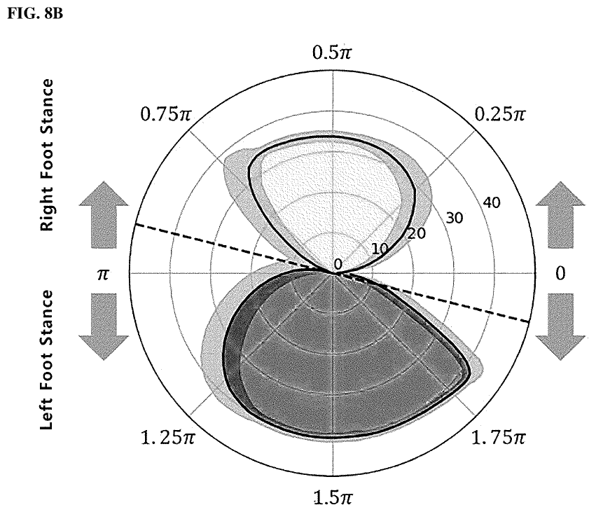

FIGS. 8A and 8B are graphs illustrating the change in the position of the center of pressure x-coordinate (COPx) in the medial-lateral direction of the human body of healthy adults and patients with gait disturbance, acquired by wearing the wearable gait disturbance analysis apparatus according to an embodiment of the present application, in the polar coordinates.

Referring to FIGS. 8A and 8B, the closed curve on the upper side representing the right foot stance with a positive value of COPx and the closed curve on the lower side representing the left foot stance with a negative value are distinguished. Looking at the polar coordinate graph for the healthy adult illustrated in FIG. 8A, it can be seen that the sizes of the areas inside the closed curves on the upper and lower sides are similar to each other, but in the polar coordinate graph for the patient with gait disturbance illustrated in FIG. 8B, the sizes of the areas inside the closed curves on the upper and lower sides are very different from each other. In other words, the ARIs of the two groups are not only very different from each other, but the position of the affected foot may also be easily distinguished with the naked eye.

The wearable gait disturbance analysis apparatus according to an embodiment of the present application may detect the movement status of the center of pressure in the human body in real time by acquiring and analyzing the ground reaction force information generated during walking using the pressure sensor installed on the bottom of the insole. In addition, the pressure sensor installed on the bottom of the insole is designed to be a low-power consumption type, and thus, can acquire bio-data for a long time, and can be applied all the time regardless of time and place.

Accordingly, the present application determines the gait soundness of the wearer and directly provides the analysis information to the PC or the smart device in real time, so it can be utilized as a low-cost, high-efficiency health management system. The present application relates to a telemedicine device that measures patient's bio-data and remotely transmits the measured patient's bio-data so that medical staff can examine the patient. The acquired data can be built into big data and utilized for the advancement of gait disturbance analysis technology in the future.

The gait cycle and the change in the position of the COPx are displayed in the polar coordinates and the ratios of the weight support of the left and right feet stances during walking are compared by drawing, thereby presenting the quantified standard for intuitively assessing gait disturbance. Therefore, it is easy to compare or distinguish gait characteristics of healthy adults and patients with gait disturbance, and it is possible to distinguish between an affected foot and a healthy foot.

In addition, according to the present application, by developing and applying a wearable gait analysis apparatus, it is possible to provide a low-cost health management system that allows anyone to easily analyze their own gait patterns and use the analyzed gait patterns as health information.

Based on the foregoing descriptions, it will be understood by those skilled in the art to which the present invention pertains that the present invention may be embodied in other specific forms without changing the technical idea or essential characteristics thereof.

Therefore, it should be understood that the embodiments described above are exemplary in all aspects and are not intended to limit the present application to the embodiments described above, and the scope of the present application is indicated by the patent claims described below rather than the detailed description described above, and all changes or modifications derived from the meaning and scope of the patent claims and equivalent concepts should be interpreted as being included in the scope of the present application.

### INDUSTRIAL APPLICABILITY

A wearable gait disturbance analysis apparatus according to one aspect of the present application may estimate a gait cycle of a pedestrian and analyze a weight shift situation according to the gait cycle to evaluate whether the gait condition is good or bad, that is, gait soundness.

The present application determines gait soundness of a wearer and directly provides analysis information to a PC or smart device in real time, so it can be utilized as a low-cost, high-efficiency health management system. The present application relates to a telemedicine device that measures patient's bio-data and remotely transmits the measured patient's bio-data so that medical staff can examine the patient. The acquired data can be built into big data and utilized for the advancement of gait disturbance analysis technology in the future.

1. A wearable gait disturbance analysis apparatus, comprising:
a plurality of left sole sensor units that measure a left ground reaction force signal generated from a left sole during walking;
a plurality of right sole sensor units that measure a right ground reaction force signal generated from a right sole during walking; and
a calculation unit that receives and calculates the left ground reaction force signal and the right ground reaction force signal,
wherein the calculation unit integrates the left ground reaction force signal and the right ground reaction force signal to calculate a center of pressure x-coordinate (COPx) in a human body in a medial-lateral direction of the human body, and uses the calculated COPx to estimate a gait cycle.

2. The wearable gait disturbance analysis apparatus of claim 1, wherein the calculation unit calculates a change in the COPx according to the estimated gait cycle, and
the wearable gait disturbance analysis apparatus further includes a gait disturbance diagnosis unit that diagnoses gait disturbance based on the change in the COPx.

3. The wearable gait disturbance analysis apparatus of claim 2, further comprising a display unit that displays the change in the COPx according to the gait cycle in polar coordinates.

4. The wearable gait disturbance analysis apparatus of claim 3, wherein the calculation unit compares areas within two closed curves on upper and lower sides, respectively, displayed on the display unit and calculates a slope of a tangent line at a contact point between the two closed curves.

5. The wearable gait disturbance analysis apparatus of claim 3, wherein the polar coordinate is represented by the closed curve on the upper side where the COPx is a positive value and the closed curve on the lower side where the COPx is a negative value, and each of the closed curve on the upper side and the closed curve on the lower side corresponds to one of a right foot stance and a left foot stance.

6. The wearable gait disturbance analysis apparatus of claim 5, wherein the calculation unit calculates an area of the closed curve on the upper side and an area of the closed curve on the lower side,
the gait disturbance diagnosis unit diagnoses the gait disturbance when a difference between the area of the closed curve on the upper side and the area of the closed curve on the lower side is greater than or equal to a preset standard, and
when the gait disturbance diagnosis unit diagnoses the gait disturbance, it is determined that a foot corresponding to the larger area of the two closed curves touches the ground longer than the other foot during walking and supports a body weight for a longer time.

7. The wearable gait disturbance analysis apparatus of claim 5, wherein the calculation unit calculates a slope of a tangent line at a contact point between the closed curve on the upper side and the closed curve on the lower side, and
the gait disturbance diagnosis unit diagnoses the gait disturbance when the slope of the tangent line is greater than or equal to a preset standard.

8. The wearable gait disturbance analysis apparatus of claim 6, wherein the calculation unit calculates an area ratio index (ARI), which is a value representing a difference in time during which each foot supports the body weight as a ratio, using the following [Equation 1], [Equation 2], [Equation 3], and [Equation 4], and
the gait disturbance diagnosis unit diagnoses whether there is the gait disturbance using the ARI as an index.
$${Area}_{Closed} = \int \frac{1}{2} {r}^{2} θ d θ \qquad [ Equation 1 ]$$
$${Area}_{ratio . R} = ( {Area}_{Closed . R} / ( {Area}_{Closed . R} + {Area}_{Closed . L} ) ) / 100 \qquad [ Equation 2 ]$$
$${Area}_{ratio . L} = ( {Area}_{Closed . L} / ( {Area}_{Closed . R} + {Area}_{Closed . L} ) ) / 100 \qquad [ Equation 3 ]$$
$$A R I = \vert {Area}_{ra tio . R} - {Area}_{ratio . L} \vert \qquad [ Equation 4 ]$$
The Area_{Closed} is an area within the closed curve, the Area_{Closed.R} and Area_{Closed.L} are an area of the closed curve corresponding to the right foot stance and an area of the closed curve corresponding to the left foot stance, respectively, the Area_{ratio.R} is a ratio of the area of the closed curve corresponding to the right foot stance to a total area of the two closed curves, and Area_{ratio.L} is a ratio of the area of the closed curve corresponding to the left foot stance to the total area of the two closed curves.

9. The wearable gait disturbance analysis apparatus of claim 8, wherein the gait disturbance diagnosis unit determines that there is the gait disturbance when the ARI value is greater than or equal to a preset standard.

10. The wearable gait disturbance analysis apparatus of claim 1, wherein the COPx in the human body is viewed in real time through a smart device.

11. The wearable gait disturbance analysis apparatus of claim 1, further comprising an amplifier that amplifies the left ground reaction force signal and the right ground reaction force signal.

12. The wearable gait disturbance analysis apparatus of claim 1, further comprising a timer that synchronizes the left ground reaction force signal and the right ground reaction force signal.

13. The wearable gait disturbance analysis apparatus of claim 1, wherein the plurality of left sole sensor units and the plurality of right sole sensor units are installed on a big toe, a 1st metatarsal bone, a 5th metatarsal bone, a cuboid bone, and a heel of the left and right feet, respectively.

14. The wearable gait disturbance analysis apparatus of claim 7, wherein the ARI is utilized as a biomarker for diagnosing a patient with the gait disturbance.
